SEQUENCE LISTING (3 sequences)

SEQ ID NO: 1 - DNA, 1494 bp, Artificial Sequence
  Lactobacillus plantarum subsp. shebah 202 16s rRNA sequence
gacgaacgctggcggcgtgcctaatacatgcaagtcgaacgaactctggtattgattggtgcttgcatcatgatttacatttgagtgagtggcgaactggtgagtaacacgtgggaaacctgcccagaagcgggggataacacctggaaacagatgctaataccgcataacaacttggaccgcatggtccgagcttgaaagatggcttcggctatcacttttggatggtcccgcggcgtattagctagatggtggggtaacggctcaccatggcaatgatacgtagccgacctgagagggtaatcggccacattgggactgagacacggcccaaactcctacgggaggcagcagtagggaatcttccacaatggacgaaagtctgatggagcaacgccgcgtgagtgaagaagggtttcggctcgtaaaactctgttgttaaagaagaacatatctgagagtaactgttcaggtattgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggctcaaccgaagaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagtatgggtagcaaacaggattagataccctggtagtccataccgtaaacgatgaatgctaagtgttggagggtttccgcccttcagtgctgcagctaacgcattaagcattccgcctggggagtacggccgcaaggctgaaactcaaaggaattgacgggggcccgcacaagcggtggagcatgtggtttaattcgaagctacgcgaagaaccttaccaggtcttgacatactatgcaaatctaagagattagacgttcccttcggggacatggatacaggtggtgcatggttgtcgtcagctcgtgtcgtgagatgttgggttaagtcccgcaacgagcgcaacccttattatcagttgccagcattaagttgggcactctggtgagactgccggtgacaaaccggaggaaggtggggatgacgtcaaatcatcatgccccttatgacctgggctacacacgtgctacaatggatggtacaacgagttgcgaactcgcgagagtaagctaatctcttaaagccattctcagttcggattgtaggctgcaactcgcctacatgaagtcggaatcgctagtaatcgcggatcagcatgccgcggtgaatacgttcccgggccttgtacacaccgcccgtcacaccatgagagtttgtaacacccaaagtcggtggggtaaccttttaggaaccagccgcctaaggtgggacagatgattagggtgaagtc

SEQ ID NO: 2 - DNA, 1494 bp, Artificial Sequence
  Lactobacillus fermentum subsp. shebah101 16s rRNA sequence
cggcggtgtgcctaatacatgcaagtcgaacgcgttggcccaattgattgatggtgcttgcacctgattgattttggtcgccaacgagtggcggacgggtgagtaacacgtaggtaacctgcccagaagcgggggacaacatttggaaacagatgctaataccgcataacaacgttgttcgcatgaacaacgcttaaaagatggcttctcgctatcacttctggatggacctgcggtgcattagcttgttggtggggtaacggcctaccaaggcgatgatgcatagccgagttgagagactgatcggccacaatgggactgagacacggcccatactcctacgggaggcagcagtagggaatcttccacaatgggcgcaagcctgatggagcaacaccgcgtgagtgaagaagggtttcggctcgtaaagctctgttgttaaagaagaacacgtatgagagtaactgttcatacgttgacggtatttaaccagaaagtcacggctaactacgtgccagcagccgcggtaatacgtaggtggcaagcgttatccggatttattgggcgtaaagagagtgcaggcggttttctaagtctgatgtgaaagccttcggcttaaccggagaagtgcatcggaaactggataacttgagtgcagaagagggtagtggaactccatgtgtagcggtggaatgcgtagatatatggaagaacaccagtggcgaaggcggctacctggtctgcaactgacgctgagactcgaaagcatgggtagcgaacaggattagataccctggtagtccatgccgtaaacgatgagtgctaggtgttggagggtttccgcccttcagtgccggagctaacgcattaagcactccgcctggggagtacgaccgcaaggttgaaactcaaaggaattgacgggggcccgcacaagcggtggagcatgtggtttaattcgaagctacgcgaagaaccttaccaggtcttgacatcttgcgccaaccctagagatagggcgtttccttcgggaacgcaatgacaggtggtgcatggtcgtcgtcagctcgtgtcgtgagatgttgggttaagtcccgcaacgagcgcaacccttgttactagttgccagcattaagttgggcactctagtgagactgccggtgacaaaccggaggaaggtggggacgacgtcagatcatcatgccccttatgacctgggctacacacgtgctacaatggacggtacaacgagtcgcgaactcgcgagggcaagcaaatctcttaaaaccgttctcagttcggactgcaggctgcaactcgcctgcacgaagtcggaatcgctagtaatcgcggatcagcatgccgcggtgaatacgttcccgggccttgtacacaccgcccgtcacaccatgagagtttgtaacacccaaagtcggtggggtaaccttttaggagccagccgcctaaggtgggacagatgattagggtgaagtc

SEQ ID NO: 3 - DNA, 1488 bp, Artificial Sequence
  Lactobacillus paraplantarum subsp. shebah401 16s rRNA sequence
acgctggcggcgtgcctaatacatgcaagtcgaacgaactctggtaatgattggtgcttgcatcatgaattacatttgagtgagtggcgaactggtgagtaacacgtgggaaacctgcccagaagcgggggataacacctggaaacagatgctaataccgcataacaacttggaccgcatggtccgagtttgaaagatggcttcggctatcacttttggatggtcccgcggcgtattagctagatggtgaggtaacggctcaccatggcaatgatacgtagccgacctgagagggtaatcggccacattgggactgagacacggcccaaactcctacgggaggcagcagtagggaatcttccacaatggacgaaagtctgatggagcaacgccgcgtgagtgaagaagggtttcggctcgtaaaactctgttgttaaagaagaacatatctgagagtaactgttcaggtattgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggctcaaccgaagaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagtatgggtagcaaacaggattagataccctggtagtccataccgtaaacgatgaatgctaagtgttggagggtttccgcccttcagtgctgcagctaacgcattaagcattccgcctggggagtacggccgcaaggctgaaactcaaaggaattgacgggggcccgcacaagcggtggagcatgtggtttaattcgaagctacgcgaagaaccttaccaggtcttgacatactatgcaaatctaagagattagacgttcccttcggggacatggatacaggtggtgcatggttgtcgtcagctcgtgtcgtgagatgttgggttaagtcccgcaacgagcgcaacccttattatcagttgccagcattaagttgggcactctggtgagactgccggtgacaaaccggaggaaggtggggatgacgtcaaatcatcatgccccttatgacctgggctacacacgtgctacaatggatggtacaacgagttgcgaactcgcgagagtaagctaatctcttaaagccattctcagttcggattgtaggctgcaactcgcctacatgaagtcggaatcgctagtaatcgcggatcagcatgccgcggtgaatacgttcccgggccttgtacacaccgcccgtcacaccatgagagtttgtaacacccaaagtcggtggggtaaccttttaggaaccagccgcctaaggtgggacagatgattagggtgaagt

(19) United States
(12) Patent Application Publication
(10) Pub. No.: US 2023/0414683 A1
(43) Pub. Date: Dec. 28, 2023

(54) ANTIOXIDANT, ANTI-INFLAMMATORY, AND WHITENING COMPOSITION COMPRISING SKIN-DERIVED LACTIC ACID BACTERIA

(71) Applicant: SHEBAH BIOTECH INC., Chuncheon-si (KR)

(72) Inventors: Yoon-Soo CHO, Seoul (KR); Hye-won LIM, Chuncheon-si (KR); Chang-jin LIM, Seoul (KR); Yu-hua HUANG, Chuncheon-si (KR); Yong-wan CHO, Chuncheon-si (KR)

(21) Appl. No.: 18/031,349

(22) PCT Filed: Oct. 12, 2021
(86) PCT No.: PCT/KR2021/014055

§ 371 Date: Apr. 12, 2023

(30) Foreign Application Priority Data
Oct. 13, 2020 (KR) 10-2020-0131716

(51) Int. Cl. A61K 35/747; A61Q 19/08; A61P 29/00; A61Q 19/02; A61K 8/99

(52) U.S. Cl. CPC A61K 35/747; A61Q 19/08; A61P 29/00; A61Q 19/02; A61K 8/99

(57) ABSTRACT

The present invention relates to an antioxidant, anti-inflammatory and whitening composition containing skin-derived lactic acid bacteria. In particular, the present invention relates to: an antioxidant, anti-inflammatory or whitening functional cosmetic composition, a food composition, a pharmaceutical composition for preventing or treating inflammation-related skin disease, and a pharmaceutical composition for preventing or treating melanin hyperpigmentation disease, which contain, as an active ingredient, a Lactobacillus plantarum subsp. shebah-202 (accession number: KCTC14087BP) strain, a Lactobacillus fermentum subsp. shebah-101 (accession number: KCTC14086BP) strain or a Lactobacillus paraplantarum subsp. shebah-401 (accession number: KCTC14088BP) strain, which is a novel lactic acid bacteria strain isolated and identified from human skin, a culture thereof, a lysate thereof, or an extract thereof.

### TECHNICAL FIELD

The present invention relates to an antioxidant, anti-inflammatory and whitening composition containing skin-derived lactic acid bacteria.

### BACKGROUND ART

The human body has a balance of substances that promote and inhibit oxidation. However, when this balance is disrupted due to various factors and tilted toward promoting oxidation, oxidative stress is induced in vivo, resulting in cell damage and pathological diseases. Reactive oxygen species (ROS), which are a direct cause of such oxidative stress, are chemically unstable and highly reactive, and thus can easily react with various biological substances such as DNA, proteins, lipids, and carbohydrates. In addition, these reactive oxygen species attack macromolecules in vivo, causing irreversible damage to cells and tissues or causing mutations, cytotoxicity, cancer and the like.

Oxidative skin aging is caused by free radicals, which are produced by the phagocytosis of leukocytes, the electron transport system during the ATP production process in mitochondria, the action of myeloperoxidase (MPO), ultraviolet rays, tobacco, normal metabolic processes, stress, pollutants, or bacteria. When radicals remain in the human body due to these causes, they cause cell destruction, connective tissue cleavage, cross-linking induction, etc. in the body, resulting in various problems such as wrinkle formation, skin cancer, cell killing, rheumatoid arthritis, atopic dermatitis, acne, etc.

In addition, in the human body, natural antioxidants (radical scavengers), such as superoxide dismutase (SOD), catalase, vitamin E, vitamin C, and ubiquinol exist which can eliminate free radicals. The functions of the body's antioxidant system gradually begin to decline due to aging, pollution, ultraviolet rays, stress, etc., and thus the antioxidant system becomes incapacitated, which eventually leads to an increase in free radicals in the body. The increased radicals destroy dermal connective tissues, such as collagen, elastin, and hyaluronic acid, causing skin subsidence (wrinkles), and destroy cells by oxidizing the lipid portion of the cell membrane, causing diseases such as dermatitis, acne, and skin cancer. In addition, these radicals are involved in a spontaneous oxidation reaction during the melanin formation process, causing spots, freckles, wrinkles, etc.

Recently, with the development of industrialization, the number of people whose skin has become sensitive due to various stimuli such as environmental pollution, stress, and ultraviolet rays has increased. Sensitive skin caused by external stimuli may be simply visible, such as erythema, but may exhibit severe and unpleasant inflammatory reactions such as itching and stinging. Thus, popular skin cosmetic compositions are burdensome to use and people are reluctant to use these compositions continuously.

In addition, human skin color is affected by environment, race, gender, etc., but is generally determined by the content of dark brown melanin present in skin, hair, eyes, etc. Melanin blocks the penetration of UV rays by absorbing more than a certain amount of UV rays, and maintains body temperature, and skin color is determined by the amount of melanin. This is not because the number of melanocytes is different, but because the size of melanocytes and the amount of melanin produced are different. Melanin absorbs more than a certain amount of UV rays and blocks harmful UV rays from penetrating into the human body, thereby protecting the human body.

In the biosynthesis of melanin, tyrosine, a type of amino acid, is oxidized by tyrosinase in the melanosomes of melanocytes to produce dihydroxyphenylalanine which then undergoes a series of enzymatic and non-enzymatic oxidation processes to form a brown or black melanin polymer. Melanosomes containing melanin granules move from the perinuclear region to the dendrite tips, migrate into the cytoplasm by phagocytosis of keratinocytes, and accumulate around the nuclei of keratinocytes. Excessive deposition of melanin in the skin can cause unwanted freckles, senile lentigo, lentigines (liver spots), melasma, brown or sunspots, sunburn pigmentation, post-inflammatory hyperpigmentation due to wounds, including abrasion and burns, or dermatitis, phototoxic reaction or other similar small, fixed pigmented lesions.

Meanwhile, lactic acid bacteria are bacteria that play a beneficial role in the human body and produce lactic acid using sugars such as carbohydrates. About 400 types of lactic acid bacteria have been discovered so far, and they are also applied to the manufacture of cosmetics. Fermentation broths obtained by culturing these lactic acid bacteria in general culture media, for example, media containing skim milk, whey, sugar, etc. as main components, have been reported to be effective in whitening, moisturizing, skin softening by an increased turnover rate of the stratum corneum, skin wrinkle relief, etc. However, in most actual cases, such functional effects of cosmetics containing these fermentation broths are exhibited through other ingredients added to cosmetics rather than by active ingredients contained in the fermentation broths.

However, a functional cosmetic for antioxidant, anti-inflammatory and whitening activities developed using lactic acid bacteria as a main ingredient has not yet been reported.

Accordingly, the present inventors have isolated and identified lactic acid bacteria derived from human skin, and have found that the identified lactic acid bacteria have excellent antioxidant, anti-inflammatory and whitening activities and may be used for the production of functional cosmetics and functional foods, thereby completing the present invention.

### DISCLOSURE

### Technical Problem

Therefore, an object of the present invention relates to an antioxidant, anti-inflammatory or whitening functional cosmetic composition containing, as an active ingredient, at least one strain selected from the group consisting of a Lactobacillus plantarum subsp. shebah-202 (accession number: KCTC14087BP) strain, a Lactobacillus fermentum subsp. shebah-101 (accession number: KCTC14086BP) strain, and a Lactobacillus paraplantarum subsp. shebah-401 (accession number: KCTC14088BP) strain (all modified later), which are skin-derived Lactobacillus sp. strains, a culture thereof, a lysate thereof, or an extract thereof.

Another object of the present invention relates to an antioxidant, anti-inflammatory or whitening food composition containing, as an active ingredient, the skin-derived Lactobacillus sp. strain of the present invention, a culture thereof, a lysate thereof, or an extract thereof.

Still another object of the present invention relates to a pharmaceutical composition for preventing or treating inflammation-related skin disease containing, as an active ingredient, the skin-derived Lactobacillus sp. strain of the present invention, a culture thereof, a lysate thereof, or an extract thereof.

Yet another object of the present invention relates to a pharmaceutical composition for preventing or treating melanin hyperpigmentation disease containing, as an active ingredient, the skin-derived Lactobacillus sp. strain of the present invention, a culture thereof, a lysate thereof, or an extract thereof.

### Technical Solution

To achieve the above-described objects of the present invention, the present invention provides an antioxidant, anti-inflammatory or whitening functional cosmetic composition containing, as an active ingredient, at least one strain selected from the group consisting of a Lactobacillus plantarum subsp. shebah-202 (accession number: KCTC14087BP) strain, a Lactobacillus fermentum subsp. shebah-101 (accession number: KCTC14086BP) strain, and a Lactobacillus paraplantarum subsp. shebah-401 (accession number: KCTC14088BP) strain, which are skin-derived Lactobacillus sp. strains, a culture thereof, a lysate thereof, or an extract thereof.

In one embodiment of the present invention, the composition may have abilities to inhibit the production of reactive oxygen species, inhibit the production of nitric oxide (NO), inhibit tyrosinase activity, inhibit melanin production, and synthesize collagen.

In one embodiment of the present invention, the composition may be formulated into one selected from the group consisting of skin lotion, skin softener, skin toner, astringent, lotion, milk lotion, moisture lotion, nourishing lotion, massage cream, nourishing cream, moisture cream, hand cream, essence, nourishing essence, pack, soap, shampoo, cleansing foam, cleansing lotion, cleansing cream, body lotion, body cleanser, emulsion, lipstick, makeup base, foundation, pressed powder, and loose powder.

The present invention also provides an antioxidant, anti-inflammatory or whitening food composition containing, as an active ingredient, at least one strain selected from the group consisting of a Lactobacillus paraplantarum subsp. shebah-202 (accession number: KCTC14087BP) strain, a Lactobacillus fermentum subsp. shebah-101 (accession number: KCTC14086BP) strain, and a Lactobacillus paraplantarum subsp. shebah-401 (accession number: KCTC14088BP) strain, which are skin-derived Lactobacillus sp. strains, a culture thereof, a lysate thereof, or an extract thereof.

In one embodiment of the present invention, the composition may have abilities to inhibit the production of reactive oxygen species, inhibit the production of nitric oxide (NO), inhibit tyrosinase activity, inhibit melanin production, and synthesize collagen.

The present invention also provides a pharmaceutical composition for preventing or treating inflammation-related skin disease containing, as an active ingredient, at least one strain selected from a Lactobacillus paraplantarum subsp. shebah-202 (accession number: KCTC14087BP) strain, a Lactobacillus fermentum subsp. shebah-101 (accession number: KCTC14086BP) strain, and a Lactobacillus paraplantarum subsp. shebah-401 (accession number: KCTC14088BP) strain, which are skin-derived Lactobacillus sp. strains, a culture thereof, a lysate thereof, or an extract thereof.

The present invention also provides a pharmaceutical composition for preventing or treating melanin hyperpigmentation disease containing, as an active ingredient, at least one strain selected from a Lactobacillus plantarum subsp. shebah-202 (accession number: KCTC14087BP) strain, a Lactobacillus fermentum subsp. shebah-101 (accession number: KCTC14086BP) strain, and a Lactobacillus paraplantarum subsp. shebah-401 (accession number: KCTC14088BP) strain, which are skin-derived Lactobacillus sp. strains, a culture thereof, a lysate thereof, or an extract thereof.

In one embodiment of the present invention, the melanin hyperpigmentation disease may be melasma, freckles, lentigo senilis, or solar lentigines.

### Advantageous Effects

The novel skin-derived lactic acid bacteria and a culture of the novel lactic acid bacteria according to the present invention have abilities to inhibit the production of reactive oxygen species, inhibit the production of nitric oxide (NO), inhibit tyrosinase activity, inhibit melanin production, and synthesize collagen, and thus may be advantageously used not only in functional cosmetics and foods having antioxidant, anti-inflammatory or whitening activity, and also for the development of agents for treating inflammation-related skin disease and melanin hyperpigmentation-related disease. In addition, these novel lactic acid bacteria do not induce cytotoxicity, and thus may be used safely without side effects.

### BRIEF DESCRIPTION OF DRAWINGS

FIG. 1 shows the results of analyzing the superoxide radical scavenging ability of the novel lactic acid bacteria isolated and identified in the present invention. In the drawings of the present invention, LP202 denotes Lactobacillus paraplantarum subsp. shebah-202, LF101 denotes Lactobacillus fermentum subsp. shebah-101, and LPP401 denotes a Lactobacillus paraplantarum subsp. shebah-401 strain.

FIG. 2 shows the results of analyzing the nitric oxide (NO) scavenging ability of the novel lactic acid bacteria isolated and identified in the present invention.

FIG. 3 shows the results of analyzing the reactive oxygen species (ROS) inhibitory ability of treatment with a culture of the novel lactic acid bacteria of the present invention after treating the mouse macrophage RAW 264.7 cell line with LPS to induce the production of reactive oxygen species (ROS).

FIG. 4 shows the results of analyzing the nitric oxide (NO) inhibitory ability of treatment with a culture of the novel lactic acid bacteria of the present invention after treating the mouse macrophage RAW 264.7 cell line with LPS to induce the production of nitric oxide (NO).

FIG. 5 shows the results of analyzing the tyrosinase activity inhibitory ability of treatment with a culture of the novel lactic acid bacteria of the present invention.

FIG. 6 shows the results of analyzing the melanin production inhibitory ability of treatment with a culture of the novel lactic acid bacteria of the present invention after treating the mouse B16F10 melanoma cell line with alpha-MSH to increase the melanin content.

FIG. 7 shows the results of analyzing collagen synthesis ability using a collagen ELISA kit after treating skin fibroblasts with a culture of the novel lactic acid bacteria of the present invention.

### BEST MODE

The present invention is characterized by providing the novel use of novel human skin-derived lactic acid bacteria having antioxidant, anti-inflammatory or whitening activity.

The present inventors have conducted studies to identify novel lactic acid bacteria that may be used as a raw material for functional cosmetics and foods having antioxidant, anti-inflammatory or whitening activity, which contain lactic acid bacteria themselves as a main component, and as a result, isolated and identified a Lactobacillus plantarum subsp. shebah-202 (accession number: KCTC14087BP) strain, a Lactobacillus fermentum subsp. shebah-101 (accession number: KCTC14086BP) strain, and a Lactobacillus paraplantarum subsp. shebah-401 (accession number: KCTC14088BP) strain, which are novel Lactobacillus sp. strains derived from human skin, and have confirmed the antioxidant, anti-inflammatory or whitening activities of the novel strains, thereby confirming that the novel strains may be used for the production of functional cosmetics and foods.

Specifically, according to one embodiment of the present invention, the present inventors isolated novel skin-derived microorganisms from obtained human skin samples and analyzed the 16s rRNA sequences of the identified strains, and as a result, identified three types of novel lactic acid bacteria that have not been previously known. These strains were named “Lactobacillus paraplantarum subsp. shebah-202”, “Lactobacillus fermentum subsp. shebah-101”, and “Lactobacillus paraplantarum subsp. shebah-401”, respectively, and deposited with the Korean Collection for Type Cultures (KCTC) on Dec. 20, 2019 under accession number KCTC14087BP for the Lactobacillus paraplantarum subsp. shebah-202 strain, accession number KCTC14086BP for the Lactobacillus fermentum subsp. shebah-101 strain, and accession number KCTC14088BP for the Lactobacillus paraplantarum subsp. shebah-401 strain.

Meanwhile, the present inventors measured the reactive oxygen species production inhibitory ability of a culture of each of the three novel strains isolated in the present invention in order to confirm the antioxidant activities of the strains, and as a result, have found that the three novel strain cultures of the present invention all have the ability to scavenge superoxide radicals and have an activity of effectively inhibiting the production of reactive oxygen species.

Thereby, the present inventors have found that the novel lactic acid bacteria of the present invention have antioxidant activity.

In another embodiment, the present inventors conducted an experiment to confirm whether the lactic acid bacteria of the present invention have anti-inflammatory activity and analyzed whether the lactic acid bacteria could inhibit nitric oxide (NO) which is an inflammation-inducing factor in vivo. As a result, the present inventors have found that cultures of the three strains all have the ability to inhibit nitric oxide and have an activity of effectively inhibiting both LPS-induced reactive oxygen species and nitric oxide in a mouse macrophage cell line.

Thereby, the present inventors have found that the novel lactic acid bacteria of the present invention have anti-inflammatory activity.

Furthermore, the present inventors conducted an experiment to confirm whether the novel lactic acid bacteria of the present invention have skin whitening activity, and analyzed whether the lactic acid bacteria have abilities to inhibit tyrosinase activity and inhibit melanin production.

As a result, the present inventors have found that cultures of the three novel strains of the present invention all have an activity of inhibiting melanin production while inhibiting tyrosinase activity.

In the biosynthesis of melanin, tyrosin, a type of amino acid, as a precursor, is biosynthesized into melanin, a polymer of indole-5,6-dihydroquinone, via DOPA, DOPA-quinone, and indole-5,6-dihydroquinone. Melanin is produced in melanocytes, and moves to and accumulates at the boundary between the epidermis and dermis in the form of granules called melanosomes. Melanin is not only a direct cause of dark skin, but also causes serious problems in terms of skin beauty, such as promoting the formation of melasma and freckles. In addition, overproduction of melanin has been found to cause skin aging and skin cancer.

In addition, tyrosinase is an enzyme that performs a key role in the melanin biosynthesis process and acts in the first stage of pigment formation. Thus, the inhibition of tyrosinase activity can induce whitening activity by inhibiting the formation of melanin pigment.

In this regard, the novel lactic acid bacteria of the present invention have both tyrosinase and melanin production inhibitory activities, and thus can induce a skin whitening effect.

Therefore, the present invention may provide a Lactobacillus paraplantarum subsp. shebah-202 (accession number: KCTC14087BP) strain, a Lactobacillus fermentum subsp. shebah-101 (accession number: KCTC14086BP) strain, or a Lactobacillus paraplantarum subsp. shebah-401 (accession number: KCTC14088BP) strain, which is a novel skin-derived Lactobacillus strain.

The present invention may also provide an antioxidant, anti-inflammatory or whitening functional cosmetic composition containing, as an active ingredient, the novel strain of the present invention, a culture thereof, a lysate thereof, or an extract thereof.

As described above, the novel lactic acid bacteria of the present invention are characterized by having abilities to inhibit the production of reactive oxygen species, inhibit the production of nitric oxide (NO), inhibit tyrosinase activity, inhibit melanin production, and synthesize collagen.

As used herein, the term “culturing” refers to any actions that are performed to grow microorganisms under appropriately artificially controlled environmental conditions. In addition, the term “culture” includes not only live cells obtained from a culture medium, but also any processed form of lactic acid bacteria known to those skilled in the art, including, for example, but not limited to, cell lysates, dried products, frozen products, etc. In one example of the present invention, a culture obtained by culturing the new strain of the present invention was used.

Furthermore, the ingredients contained in the cosmetic composition of the present invention include, in addition to the novel strain of the present invention, a culture thereof, a lysate thereof, or an extract thereof as an active ingredient, ingredients commonly used in cosmetic compositions, for example, conventional adjuvants and carriers, such as an antioxidant, a stabilizer, a solubilizer, a vitamin, a pigment and a flavor.

The cosmetic composition of the present invention may be prepared in any formulation commonly prepared in the art, and for example, may be formulated into a solution, a suspension, an emulsion, a paste, a gel, a cream, a lotion, powder, soap, a surfactant-containing cleanser, oil, a powder foundation, an emulsion foundation, a wax foundation, a spray, etc., without being limited thereto. More specifically, the cosmetic composition of the present invention may be prepared in the formulation of skin lotion, skin softener, skin toner, astringent, lotion, milk lotion, moisture lotion, nourishing lotion, massage cream, nourishing cream, moisture cream, hand cream, essence, nourishing essence, pack, soap, shampoo, cleansing foam, cleansing lotion, cleansing cream, body lotion, body cleanser, emulsion, lipstick, makeup base, foundation, pressed powder, and loose powder.

When the formulation of the present invention is a paste, cream, or gel, animal oil, vegetable oil, wax, paraffin, starch, tragacanth, a cellulose derivative, polyethylene glycol, silicone, bentonite, silica, talc, zinc oxide or the like may be used as a carrier component. When the formulation of the present invention is a solution or an emulsion, a solvent, a solubilizing agent, or an emulsifying agent may be used as a carrier component, and examples thereof include water, ethanol, isopropanol, ethyl carbonate, ethyl acetate, benzyl alcohol, benzyl benzoate, propylene glycol, 1,3-butylglycol oil, glycerol aliphatic ester, polyethyleneglycol, or fatty acid ester of sorbitan. When the formulation of the present invention is a suspension, a liquid diluent such as water, ethanol, or propylene glycol, a suspending agent such as ethoxylated isostearyl alcohol, polyoxyethylene sorbitol ester, or polyoxyethylene sorbitan ester, microcrystalline cellulose, aluminum metahydroxide, bentonite, agar, tragacanth, or the like may be used as a carrier component. When the formulation of the present invention is powder or a spray, lactose, talc, silica, aluminum hydroxide, calcium silicate, or polyamide powder may be used as a carrier component. In particular, when the formulation is a spray, it may further contain a propellant such as chlorofluorohydrocarbon, propane/butane, or dimethyl ether. When the formulation of the present invention is a surfactant-containing cleanser, aliphatic alcohol sulfate, aliphatic alcohol ether sulfate, sulfosuccinic acid monoester, isethionate, an imidazolium derivative, methyltaurate, sarcosinate, fatty acid amide ether sulfate, alkylamidobetaine, aliphatic alcohol, fatty acid glyceride, fatty acid diethanolamide, vegetable oil, a lanolin derivative, ethoxylated glycerol fatty acid ester, or the like may be used as a carrier component.

When the cosmetic composition of the present invention is soap, a surfactant-containing cleansing formulation, or a surfactant-free cleansing formulation, it may be applied to the skin and then wiped off, removed, or washed off with water. As a specific example, the soap is liquid soap, powder soap, solid soap, or oil soap, and the surfactant-containing cleansing formulation is a cleansing foam, cleansing water, a cleansing towel, or a cleansing pack, and the surfactant-free cleansing formulation is cleansing cream, cleansing lotion, cleansing water, or cleansing gel, without being limited thereto.

Furthermore, the present invention may provide an antioxidant, anti-inflammatory or whitening food composition containing, as an active ingredient, the novel strain of the present invention, a culture thereof, a lysate thereof, or an extract thereof.

The three novel strains identified in the present invention are lactic acid bacteria belonging to the genus Lactobacillus plantarum, the genus Lactobacillus fermentum, and the genus Lactobacillus paraplatanum, respectively, and these lactic acid bacteria have probiotic activity, and thus may be ingested into the body.

Therefore, the novel strains of the present invention may be used not only as functional cosmetic compositions having antioxidant, anti-inflammatory or whitening activity, but also as functional food compositions having antioxidant, anti-inflammatory or whitening activity.

The food composition of the present invention includes any types of food compositions, such as functional foods, nutritional supplements, health foods, and food additives. The above types of food composition may be produced in various forms according to the conventional methods known in the art. For example, as for health foods, the novel strain itself of the present invention, a lysate thereof, and a culture thereof may be produced in the forms of a tea, a juice, and a drink so as to be ingested, or may be granulated, encapsulated, or powdered so as to be ingested. In addition, at least one of the novel strain of the present invention, a lysate thereof, and a culture thereof may be prepared in the form of a composition by mixing the same with a known substance or active ingredient known to have an anti-wrinkle effect or an effect of improving skin barrier function. In addition, functional foods may be produced by adding at least one of the novel strain of the present invention, a lysate thereof, and a culture thereof to beverages (including alcoholic beverages), fruits and processed foods thereof (e.g., canned fruit, bottled food, jam, marmalade, etc.), fishes, meats and processed products thereof (e.g., ham, sausage, corned beef, etc.), breads, noodles (e.g., udong, buckwheat noodles, ramen, spaghetti, macaroni, etc.), fruit juices, a variety of drinks, cookies, Korean hard taffy, dairy products (e.g., butter, cheese, etc.), edible vegetable oils, margarine, vegetable proteins, retort foods, frozen foods, various seasonings (e.g., soybean paste, soybean sauce, sauces, etc.), or the like.

The preferred content of the novel strain of the present invention, a lysate thereof, or a culture thereof in the food composition of the present invention is preferably 0.01 to 50 wt % based on the weight of the finally produced food, without being limited thereto. In addition, at least one selected from the group consisting of the novel strain of the present invention, a lysate thereof, and a culture thereof may be produced in the form of powder or concentrate so that it may be used in the form of a food additive as an active ingredient.

Furthermore, the present invention may provide a pharmaceutical composition for preventing or treating inflammation-related skin disease containing, as an active ingredient, at least one strain selected from the group consisting of a Lactobacillus paraplantarum subsp. shebah-202 (accession number: KCTC14087BP) strain, a Lactobacillus fermentum subsp. shebah-101 (accession number: KCTC14086BP) strain, and a Lactobacillus paraplantarum subsp. shebah-401 (accession number: KCTC14088BP) strain, which are skin-derived Lactobacillus sp. strains, a culture thereof, a lysate thereof, or an extract thereof.

As described above, the novel lactic acid bacteria of the present invention have excellent anti-inflammatory activity for skin cells, and thus may exhibit an effect of preventing, alleviating or treating inflammation-related skin diseases that may be caused by skin inflammation.

Examples of the inflammation-related skin diseases according to the present invention include, but are not limited to, atopic dermatitis, contact dermatitis, seborrhea, and acne.

The present invention may also provide a pharmaceutical composition for preventing or treating melanin hyperpigmentation disease containing, as an active ingredient, at least one strain selected from the group consisting of a Lactobacillus paraplantarum subsp. shebah-202 (accession number: KCTC14087BP) strain, a Lactobacillus fermentum subsp. shebah-101 (accession number: KCTC14086BP) strain, and a Lactobacillus paraplantarum subsp. shebah-401 (accession number: KCTC14088BP) strain, which are skin-derived Lactobacillus sp. strains, a culture thereof, a lysate thereof, or an extract thereof.

As used herein, the term “melanin hyperpigmentation” means that a specific portion of the skin or nail becomes swarthier or darker than other portions due to an excessive increase in melanin.

Examples of the melanin hyperpigmentation disease include, but are not limited to, melasma, freckles, lentigo senilis, solar lentigines, and the like.

As used herein, the term “pharmaceutically effective amount” refers to an amount sufficient to treat a disease at a reasonable benefit/risk ratio applicable to any medical treatment. The pharmaceutically effective amount may be determined depending on factors, including the kind and severity of the subject's disease, the activity of the drug, sensitivity to the drug, the time of administration, the route of administration, excretion rate, the period of treatment, and drugs used in combination with the composition, as well as other factors well known in the medical field.

The pharmaceutical composition of the present invention may be prepared using pharmaceutically suitable and physiologically acceptable adjuvants in addition to the active ingredient of the present invention, and examples of the adjuvants include excipients, disintegrants, sweeteners, binders, coating agents, swelling agents, lubricants, glidants, and flavoring agents.

For administration, the pharmaceutical composition may preferably be formulated as a pharmaceutical composition containing one or more pharmaceutically acceptable carriers in addition to the active ingredient of the present invention.

Dosage forms of the pharmaceutical composition may be granules, powders, tablets, coated tablets, capsules, suppositories, solutions, syrups, juices, suspensions, emulsions, drops, or injectable solutions. For example, for oral administration in the form of a tablet or capsule, the active ingredient may be combined with an oral, non-toxic pharmaceutically acceptable inert carrier such as ethanol, glycerol, or water. In addition, if desired or necessary, suitable binders, lubricants, disintegrants and coloring agents may likewise be incorporated into the mixture. Suitable binders include, but are not limited to, starch, gelatin, natural sugars, such as glucose or beta-lactose, sweeteners made from maize, natural and synthetic rubber, such as acacia, tragacanth or sodium oleate, sodium stearate, magnesium stearate, sodium benzoate, sodium acetate, sodium chloride, and the like. The disintegrants include, but are not limited to, starch, methylcellulose, agar, bentonite, xanthan gum, and the like. A pharmaceutically acceptable carrier for a composition formulated as a liquid solution may be suitable for sterilization and administration in vivo, and may be saline, sterilized water, Ringer solution, buffered saline, albumin injection solution, dextrose solution, malto-dextrin solution, glycerol, ethanol, and a mixture including at least one of these components. If necessary, other conventional additives, such as an antioxidant, a buffer, and a bacteriostatic agent may be added. In addition, a diluent, a dispersant, a surfactant, a binder, and a lubricant may be further added to prepare injectable formulations such as aqueous solutions, suspensions, emulsions, or the like, pills, capsules, granules, or tablets. Furthermore, the pharmaceutical composition may be preferably formulated depending on each disease or ingredient using the method disclosed in Remington's Pharmaceutical Science, Mack Publishing Company, Easton PA, which is a suitable method known in the related art.

In one embodiment of the present invention, the active ingredient of the present invention may be contained in an amount of 0.001 to 99 wt % based on the total weight of the composition.

Furthermore, the present inventors analyzed the anti-inflammatory, antioxidant and whitening activities of a mixture of cultures of the novel lactic acid bacteria strains of the present invention, and as a result, confirmed that the anti-inflammatory, antioxidant and whitening activities were better in the group treated with a mixture of the three strain cultures mixed at a ratio of 1:1:1 than in the groups treated with these strain cultures alone, and the efficacies of mixtures of the three strain cultures mixed at other ratios were lower than the mixture of the three strain cultures mixed at a ratio of 1:1:1.

Hereinafter, the present invention will be described in more detail with reference to examples. These examples are intended to explain the present invention in more detail, and the scope of the present invention is not limited to these examples.

### Example 1

### Isolation and Identification of New Strains Derived from Human Skin

### <1-1>Sample Collection and Lactic Acid Bacteria Isolation

Under the consent of 30 subjects living in Chuncheon, Korea, skin surface microorganisms on both facial cheeks, foreheads and palms were swabbed at constant temperature and humidity (temperature: 22±2° C., and humidity: 50±5%). The swabbing was performed using a Quick Swab kit (3M Microbiology, USA), and each sample was collected from a surface area of about 12 cm² for each test subject according to the swabbing method recommended by the swab kit manufacturer. Each swabbed sample was streaked on a solid agar plate containing agar using a flame-sterilized platinum loop, followed by culturing at a temperature of 37° C. for 3 days until single colonies appeared. Next, 50 individual single colonies were collected, and then streaked and subcultured on de Man, Rogosa and Sharpe (MRS) agar plates to increase the growth activities of the strains. The single colonies cultured according to the above process were isolated, and the 16s RNA sequences of the isolated strains were analyzed.

### <1-2>Identification of New Strains by Sequencing

In order to identify the strains isolated in Example 1-1, 16S rRNA sequencing was performed. For sequencing, genomic DNA was isolated using a chromosomal DNA extraction kit, and the obtained gDNA was amplified by PCR using universal primers [27F: 5′ AGA GTT TGA TCM TGG CTC AG 3′, and 1492R: 5′ GGT TAC CTT GTT ACG ACT TC 3′] and sequenced. Homology analysis for the results obtained by 16s rRNA sequencing was performed using the NCBI BLAST database search tool.

As a result of sequencing, it could be seen that the lactic acid bacteria isolated in the present invention were novel lactic acid bacteria that have not been known, and as a result of sequence homology analysis, it was shown that the new lactic acid bacteria of the present invention had a sequence homology of at least 90% to the genus Lactobacillus.

Accordingly, the three novel lactic acid bacteria strains isolated in the present invention were named “Lactobacillus paraplantarum subsp. shebah-202”, “Lactobacillus fermentum subsp. shebah-101”, and “Lactobacillus paraplantarum subsp. shebah-401”, respectively, and deposited with the Korean Collection for Type Cultures (KCTC) on Dec. 20, 2019 under accession number KCTC14087BP for the Lactobacillus paraplantarum subsp. shebah-202 strain, accession number KCTC14086BP for the Lactobacillus fermentum subsp. shebah-101 strain, and accession number KCTC14088BP for the Lactobacillus paraplantarum subsp. shebah-401 strain.

In addition, the 16s rRNA sequences of these novel strains are set forth in SEQ ID NOs: 1 to 3. Specifically, the 16s rRNA sequence of Lactobacillus paraplantarum subsp. shebah-202 is set forth in SEQ ID NO: 1, the 16s rRNA sequence of Lactobacillus fermentum subsp. shebah-101 is set forth in SEQ ID NO: 2, and the 16s rRNA sequence of Lactobacillus paraplantarum subsp. shebah-401 is set forth in SEQ ID NO: 3.

In addition, the sequences of these novel strains identified in the present invention were deposited in the NCBI GenBank sequence database under accession numbers MN625238.1 (Lactobacillus paraplantarum strain shebah-202 16S ribosomal RNA gene, partial sequence), MN625236.1 (Lactobacillus fermentum strain shebah-101 16S ribosomal RNA gene, partial sequence) and MN602521.1 (Lactobacillus paraplantarum strain shebah-401 16S ribosomal RNA gene, partial sequence), respectively.

### Example 2

### Preparation of Cultures of Novel Strains

For the three novel lactic acid bacteria strains isolated and identified in Example 1, strain cultures were obtained through the following method. First, single colonies were collected from the MRS agar plates, and then inoculated into MRS liquid media and cultured at 100 rpm and a temperature of 37° C. with shaking in a shaking incubator. After subculturing 3 times, main culturing was performed for 5 days. Thereafter, the cultures were centrifuged to collect supernatants, and then the supernatants were filtered through a 0.2-μm pore size filter to obtain a culture of each of Lactobacillus paraplantarum subsp. shebah-202 (LP202), Lactobacillus fermentum subsp. shebah-101 (LF101) and Lactobacillus paraplantarum subsp. shebah-401 (LPP401), which are novel strains.

### Example 3

### Analysis of Antioxidant Activities of Novel Strains

Superoxide radical, known as a reactive oxygen species (ROS), is a precursor of other reactive oxygen species, and the production of other reactive oxygen species can be inhibited by scavenging the superoxide radical. Accordingly, the present inventors conducted an experiment to confirm whether the three lactic acid bacteria strains identified in the present invention exhibited antioxidant activity by scavenging superoxide radicals.

To this end, 20 μL of a mixture of each strain culture (sample) prepared in Example 2 and 0.2 M Tris-HCl was added to a mixed solution of 156 μM NADH (60 μL), 100 μM nitroblue tetrazolium (60 μL) and 20 μM phenazine methosulfate (60 μL), and then absorbance at 560 nm was measured.

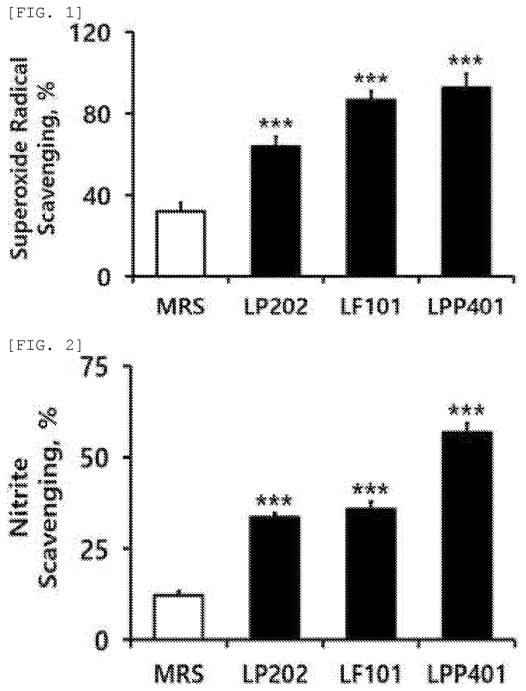

As a result, as shown in FIG. 1, it was confirmed that the three novel lactic acid bacteria strains of the present invention all had superoxide radical scavenging ability, and in particular, the Lactobacillus paraplantarum subsp. shebah-401 strain had the best antioxidant activity.

### Example 4

### Analysis of Anti-Inflammatory Activities of Novel Strains

### <4-1>Analysis of Nitric Oxide (NO) Scavenging Ability

Nitric oxide (NO) is a mediator that induces inflammation in vivo, and is known to be produced in the process in which L-arginine is degraded into L-citrulline by inducible NO synthase (iNOS). Studies have been actively conducted on natural anti-inflammatory products that can effectively remove NO that causes inflammation in the human body. Since NO exists in equilibrium with nitrite in an aqueous solution, the level of NO can be determined by quantifying the level of nitrite, and the NO scavenging ability can be estimated through the nitrite scavenging ability. Furthermore, since the nitrite scavenging ability can be a means for confirming the anti-inflammatory effect of the sample, the present inventors analyzed the anti-inflammatory activities by measuring the nitrite scavenging abilities of the three lactic acid bacteria strains of the present invention as follows.

To this end, 30 μL of 0.1 M citrate buffer (pH 3.0) and 6 μL of 50 μg/mL NaNO₂ were mixed together, and then 60 μL of a mixed solution of each strain culture (sample) prepared in Example 2 and 0.1 M citrate buffer (pH 3.0), mixed with 150 μL of Griess reagent, was added thereto, and then the absorbance at 538 nm was measured.

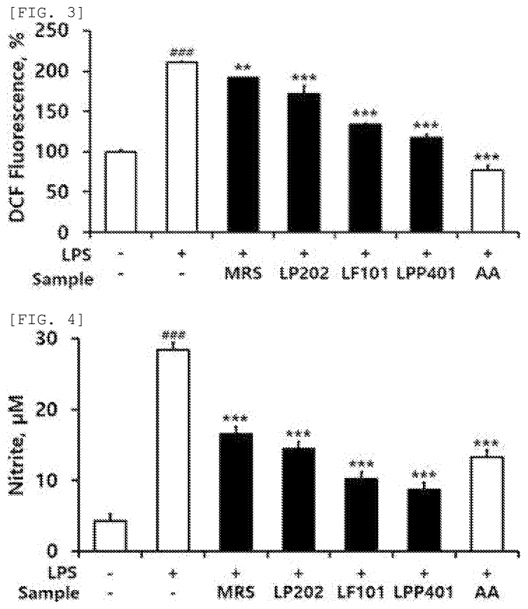

As a result, as shown in FIG. 2, it was confirmed that the three novel lactic acid bacteria strains of the present invention had nitrite scavenging ability, and in particular, the Lactobacillus paraplantarum subsp. shebah-401 strain had the best anti-inflammatory activity.

### <4-2>Analysis of the Ability to Inhibit Reactive Oxygen Species (ROS) that was Increased by LPS in RAW 264.7 Cell Line

The mouse macrophage RAW 264.7 cell line was treated with lipopolysaccharide (LPS) as an inflammatory response inducer, to increase the level of ROS, and then the effect of each novel lactic acid bacteria strain of the present invention on the increased ROS level was analyzed.

To this end, RAW 264.7 cells were cultured in a 6-well plate at a cell density of 2×10⁵ cells/well for 24 hours, and then pretreated with 10 μL of the culture (sample) of each lactic acid bacteria strain of the present invention for 30 minutes, and treated with LPS (10 ng/mL) for 24 hours. Next, the supernatant was removed, and then the remaining cells were washed twice with 1 mL/well of 1× PBS, and then treated with 500 μL/well of a solution of 100 μM DCFH-DA in 1× PBS. After 30 minutes of incubation in an incubator at 37° C. under CO₂, the cells were washed twice with 1 mL/well of 1× PBS and collected with 500 μL/well of 1× PBS, and 200 μL was loaded into each well of a 96-well plate, and fluorescence was measured with a multi-mode microplate reader (excitation: 488 nm; emission: 535 nm).

As a result, as shown in FIG. 3, it was confirmed that, when the RAW 264.7 cells were treated with LPS, the ROS level significantly increased by more than 2 times compared to that in the group not treated with LPS, whereas the increased ROS level was significantly decreased by treatment with the culture of each novel lactic acid bacteria strain of the present invention. In addition, it was confirmed that the effect of inhibiting LPS-induced ROS production was higher in the order of LP202<LF101<LPP401, indicating that the LPP401 strain had the best effect of inhibiting ROS production.

### <4-3>Analysis of Nitric Oxide (NO) Inhibitory Ability in RAW 264.7 Cell Line

Nitric oxide (NO) is a mediator that induces inflammation in vivo, and is known to be produced in the process in which L-arginine is degraded into L-citrulline by inducible NO synthase (iNOS). It is known that the inhibition of NO production can alleviate dermatitis.

Accordingly, the present inventors cultured RAW 264.7 cells in a 6-well plate at a cell density of 2×10⁵ cells/well for 24 hours, and then pretreated with 10 μL of each of the cultures of the three novel lactic acid bacteria strains of the present invention for 30 minutes. Next, the cells were treated with LPS (10 ng/mL) for 24 hours, and then 500 μL of each culture supernatant was taken to measure the nitrite level. 50 μL of Griess reagent was added to 50 μL of the RAW 264.7 cell culture supernatant which was then incubated at room temperature for 10 minutes, and then the absorbance at 540 nm was measured using a spectrophotometer. The nitrite level was determined by substituting the total amount of nitrite into a standard curve obtained after measuring absorbance in the same way as described above using NaNO₂ (0 μM to 64 μM) solution.

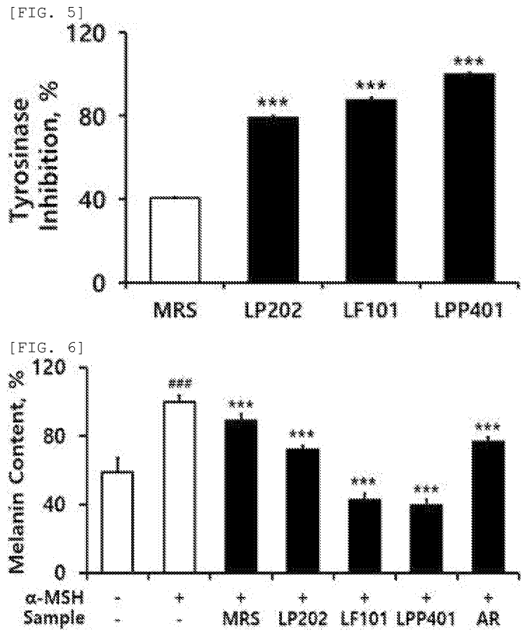

As a result, as shown in FIG. 4, it was confirmed that the nitrite level that was increased by treating RAW 264.7 with LPS was significantly decreased when the cells were treated with the culture of each strain of the present invention. From these results, the present inventors could see that the novel lactic acid bacteria strains of the present invention all had excellent anti-inflammatory activity.

### Example 5

### Analysis of Whitening Activities of Novel Strains

### <5-1>Tyrosinase Inhibitory Activity

Regarding the inhibitory effect on the DOPA oxidase activity that converts 3,4-dihydroxy-L-phenyalanine (L-DOPA) into L-dopaquinone, among the two activities of tyrosinase known to catalyze the rate-limiting step of the melanin production pathway, the whitening activity of each novel strain of the present invention was analyzed.

To this end, the reaction product L-dopaquinone resulting from the oxidation of L-DOPA as a substrate by purified mushroom tyrosinase was measured spectroscopically. First, 20 μL of a mixture of the culture of each novel strain of the present invention and PBS was added to 2 μL of a mushroom tyrosinase solution prepared at a concentration of 7,500 U/mL by dissolving 138 μL of 1× PBS (pH 7.4) and the enzyme mushroom tyrosinase in 1× PBS (pH 7.4), followed by pre-incubation at 37° C. for 90 minutes. Thereafter, 40 μL of a solution prepared by dissolving L-DOPA in 1× PBS (pH 7.4) at a concentration of 2.5 mM was added to the pre-incubated solution, and the absorbance at 450 nm was measured.

As a result, as shown in FIG. 5, it was confirmed that the three types of novel lactic acid bacteria of the present invention all had tyrosinase inhibitory activity, and the LPP401 strain had the best tyrosinase inhibitory activity.

### <5-2>Analysis of Effect on Melanin Content of Melanocytes

An important factor that determines the color of human skin is melanin, which is produced in melanocytes and migrates to other skin tissues such as the epidermis. Therefore, the inhibition of melanin production in melanocytes has a skin whitening effect.

Accordingly, the present inventors measured the degree of melanin content reduction caused by treatment with each strain of the present invention under the condition of increasing the melanin content by treating the B16F10 mouse melanoma cell line with alpha-melanocyte-stimulating hormone (alpha-MSH).

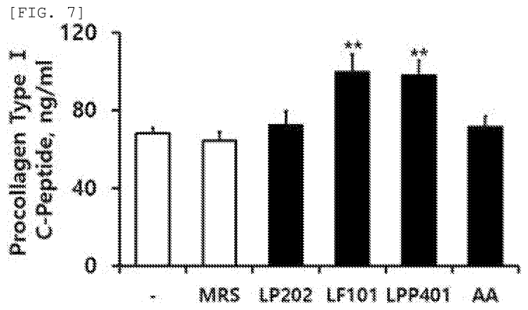

As a result, as shown in FIG. 6, it was confirmed that the melanin content that was increased by treatment with alpha-MSH treatment was significantly inhibited in the groups treated with the cultures of the three novel lactic acid bacteria strains of the present invention, and in particular, the LF101 and LPP401 strains exhibited very good melanin inhibitory activity.

### Example 6

### Analysis of Collagen Synthesis Ability of Novel Strains in Skin Fibroblasts

It is known that collagen synthesis in skin fibroblasts is closely related to skin wrinkle formation, and thus a decrease in collagen synthesis leads to an increase in wrinkle formation. Accordingly, the present inventors conducted an experiment to confirm whether each novel lactic acid bacteria strain of the present invention could promote collagen synthesis in skin fibroblasts.

To this end, NHDF cells (5×10⁵ cells/well) were seeded into a 6-well plate and cultured for 24 hours. Next, the NHDF cells were treated with the culture of each novel lactic acid bacteria strain of the present invention and then cultured for 48 hours. The supernatant was collected, and then the collagen content was quantified using a collagen ELISA kit.

As a result, as shown in FIG. 7, it was confirmed that the synthesis of collagen significantly increased in all the groups treated with the cultures of the three novel lactic acid bacteria strains of the present invention.

From the above results, the present inventors could see that the Lactobacillus paraplantarum subsp. shebah-202 (accession number: KCTC14087BP) strain, the Lactobacillus fermentum subsp. shebah-101 (accession number: KCTC14086BP) strain, and the Lactobacillus paraplantarum subsp. shebah-401 (accession number: KCTC14088BP) strain, which are the novel lactic acid bacteria strains isolated and identified in the present invention, all have excellent antioxidant activity, excellent anti-inflammatory activity, excellent skin whitening activity, and an effect of reducing wrinkles by collagen synthesis, and thus may be advantageously used in the production of functional cosmetics and functional foods.

So far, the present invention has been described with reference to the embodiments. Those of ordinary skill in the art to which the present invention pertains will appreciate that the present invention may be embodied in modified forms without departing from the essential characteristics of the present invention. Therefore, the disclosed embodiments should be considered from an illustrative point of view, not from a restrictive point of view. The scope of the present invention is defined by the claims rather than the foregoing description, and all differences within the scope equivalent thereto should be construed as being included in the present invention.

Name of depository authority: the Korean Collection for Type Cultures at the Korea Research Institute of Bioscience and Biotechnology

Address of depository authority: 181 Ipsin-gil (Sinjeong-dong), Jeongeup-si, Jeollabuk-do, 56212, Republic of Korea

Accession number: KCTC14086BP

Deposit date: Dec. 20, 2019

Name of depository authority: the Korean Collection for Type Cultures at the Korea Research Institute of Bioscience and Biotechnology

Address of depository authority: 181 Ipsin-gil (Sinjeong-dong), Jeongeup-si, Jeollabuk-do, 56212, Republic of Korea

Accession number: KCTC14087BP

Deposit date: Dec. 20, 2019

Name of depository authority: the Korean Collection for Type Cultures at the Korea Research Institute of Bioscience and Biotechnology

Address of depository authority: 181 Ipsin-gil (Sinjeong-dong), Jeongeup-si, Jeollabuk-do, 56212, Republic of Korea

Accession number: KCTC14088BP Deposit date: Dec. 20, 2019

1. An antioxidant, anti-inflammatory or whitening functional cosmetic composition containing, as an active ingredient, at least one strain selected from the group consisting of a Lactobacillus paraplantarum subsp. shebah-202 (accession number: KCTC14087BP) strain, a Lactobacillus fermentum subsp. shebah-101 (accession number: KCTC14086BP) strain, and a Lactobacillus paraplantarum subsp. shebah-401 (accession number: KCTC14088BP) strain, which are skin-derived Lactobacillus sp. strains, a culture thereof, a lysate thereof, or an extract thereof.

2. The antioxidant, anti-inflammatory or whitening functional cosmetic composition of claim 1, wherein the composition has abilities to inhibit reactive oxygen species production, inhibit nitric oxide (NO) production, inhibit tyrosinase activity, inhibit melanin production, and synthesize collagen.

3. The antioxidant, anti-inflammatory or whitening functional cosmetic composition of claim 1, wherein the composition is formulated into one selected from the group consisting of skin lotion, skin softener, skin toner, astringent, lotion, milk lotion, moisture lotion, nourishing lotion, massage cream, nourishing cream, moisture cream, hand cream, essence, nourishing essence, pack, soap, shampoo, cleansing foam, cleansing lotion, cleansing cream, body lotion, body cleanser, emulsion, lipstick, makeup base, foundation, pressed powder, and loose powder.

4. An antioxidant, anti-inflammatory or whitening food composition containing, as an active ingredient, at least one strain selected from the group consisting of a Lactobacillus paraplantarum subsp. shebah-202 (accession number: KCTC14087BP) strain, a Lactobacillus fermentum subsp. shebah-101 (accession number: KCTC14086BP) strain, and a Lactobacillus paraplantarum subsp. shebah-401 (accession number: KCTC14088BP) strain, which are skin-derived Lactobacillus sp. strains, a culture thereof, a lysate thereof, or an extract thereof.

5. The antioxidant, anti-inflammatory or whitening food composition of claim 4, wherein the composition has abilities to inhibit reactive oxygen species production, inhibit nitric oxide (NO) production, inhibit tyrosinase activity, inhibit melanin production, and synthesize collagen.

6. A pharmaceutical composition for preventing or treating inflammation-related skin disease containing, as an active ingredient, at least one strain selected from the group consisting of a Lactobacillus paraplantarum subsp. shebah-202 (accession number: KCTC14087BP) strain, a Lactobacillus fermentum subsp. shebah-101 (accession number: KCTC14086BP) strain, and a Lactobacillus paraplantarum subsp. shebah-401 (accession number: KCTC14088BP) strain, which are skin-derived Lactobacillus sp. strains, a culture thereof, a lysate thereof, or an extract thereof.

7. A pharmaceutical composition for preventing or treating melanin hyperpigmentation disease containing, as an active ingredient, at least one strain selected from the group consisting of a Lactobacillus paraplantarum subsp. shebah-202 (accession number: KCTC14087BP) strain, a Lactobacillus fermentum subsp. shebah-101 (accession number: KCTC14086BP) strain, and a Lactobacillus paraplantarum subsp. shebah-401 (accession number: KCTC14088BP) strain, which are skin-derived Lactobacillus sp. strains, a culture thereof, a lysate thereof, or an extract thereof.

8. The pharmaceutical composition of claim 7, wherein the melanin hyperpigmentation disease is melasma, freckles, lentigo senilis, or solar lentigines.
